(19) United States
(12) Patent Application Publication
(10) Pub. No.: US 2023/0128706 A1
(43) Pub. Date: Apr. 27, 2023

(54) System and Method for Treating Sleep Apnea, Night-time Hearing Impairment and Tinnitus With Acoustic Neuromodulation

(71) Applicant: Whispersom Corp., Annapolis, MD (US)

(72) Inventors: Michael G. NATHANS, Annapolis, MD (US); David G. GOLDSTEIN, Groton, MA (US); Craig F. FEIED, Honolulu, HI (US); Kevin E. GOLDSTEIN, Marana, AZ (US)

(73) Assignee: Whispersom Corp., Annapolis, MD (US)

(21) Appl. No.: 18/048,675

(22) Filed: Oct. 21, 2022

Related U.S. Application Data
(60) Provisional application No. 63/270,677, filed on Oct. 22, 2021.

(51) Int. Cl. A61F 11/00; A61M 21/02

(52) U.S. Cl. CPC A61F 11/00; A61M 21/02; A61M 2021/0027

(57) ABSTRACT

A method and device for treating tinnitus and/or hearing impairment of a user at bedtime includes providing an audio stimulus to the user using a parametric array speaker. Some embodiments treat tinnitus by removing a frequency notch corresponding to the user's tinnitus frequencies from the audio signal. The audio signal may be ambient sound detected by a microphone, or may be a prerecorded file which may comprise noise or other audio such as audiobooks or music. Some embodiments combine treatment of tinnitus or hearing impairment with treatment for apnea, hypopnea or snoring.

### CROSS-REFERENCE TO RELATED APPLICATIONS

This application claims priority to U.S. Provisional Application No. 63/270,677 filed Oct. 22, 2021. The above application is incorporated by reference in its entirety.

### FIELD OF THE INVENTION

The disclosure relates to a method and system for the bed-time delivery of a variety of audio content including therapeutic treatment for sleep apnea, hypopnea, snoring, night-time hearing impairment, and/or tinnitus.

### BACKGROUND OF THE INVENTION

The importance of sleep for an individual's overall health has become well understood in recent years. Enhanced sleep has been linked to improved cognitive outcomes such as enhanced memory consolidation (Ngo, Martinetz, Born, & Molle, 2013; Ong et al., 2016), whereas insufficient sleep is associated with poorer physical health outcomes (Kecklund & Axelsson, 2016). Disrupted and poor-quality sleep is associated with numerous deleterious health outcomes, including cardiovascular and metabolic diseases (Diekelmann & Born, 2010).

A number of studies have determined that hearing loss and/or tinnitus have an adverse impact on the ability of the user to both fall and stay asleep.

Hearing impairment (HI) is a permanent reduction in unaided hearing thresholds and is the most common sensory impairment, representing a significant global disease burden (Correia et 2016; Pascolini & Smith, 2010). Disrupted sleep through HI may therefore indirectly contribute to worsening of cognitive performance and disease outcomes in those with HI. (Luyster et al., 2012).

HI may also adversely impact sleep by causing or increasing anxiety. Anxiety may be increased through anticipation or experience of communication difficulties in challenging work or social environment, a negative psychological state that is already known to negatively impact sleep (Danermark & Gellerstedt, 2004; Staner, 2003). Monzani et al. (2008) suggest that anxiety and its impact on psychological well-being may be due to a fear of communicating in acoustically challenging situations and environments, which are likely a constant consideration for HI adults when working or socializing.

The absence of [ambient] acoustic stimuli has also been shown to alter sleep (Velluti, 2018). Evidence suggests that HI is associated with specific alterations to sleep architecture (i.e., the cyclic structure of sleep throughout the night). Reported alterations include increased overall sleep duration and altered EEG measures of sleep architecture compared to controls, including alterations to the amount of time spent in various sleep stages, and increased SS [Slow-wave Sleep] frequency and duration (Nakajima et al., 2014; Nakayama et al., 2010; Scrofani et al., 110106; Velluti et al., 2010).

Neuroplastic alterations to sleep were suggested by Velluti et al. (2010) as a consequence of changes to auditory input. The authors reported a statistically significant difference in sleeping EEG results in cochlear implant users between device “on” and “off” conditions. This study provides quasi-experimental evidence for reduced auditory input altering sleep structure. Velluti et al. (2010) also report increases in amounts of REM sleep in participants deprived of night-time sound. Taken together, these results suggest neural plasticity through diminished auditory input may result in negative alterations to sleep patterns.

Almost 100 percent of tinnitus cases occur with an underlying hearing loss. Among 222.1±3.4 million American adults, 21.4±3.4 million (10.6±0.3%) experienced tinnitus in the past 12 months. Among tinnitus sufferers, 27.0% had symptoms lasting longer than 15 years; 36.0% had near constant symptoms. Higher rates of tinnitus were reported in those with consistent work (OR 3.3; CI: 2.10-3.7) and recreational time (OR 2.6; CI: 2.3-2.10) with loud noise exposure. Years of work-related noise exposure correlated with increasing prevalence of tinnitus r=0.130 (105% CI, 0.103 to 0.157). 7.2% reported their tinnitus as a “big” or “very big” problem versus 41.6% reporting it as a “small” problem (Bhatt et. al. 2016).

Some people with tinnitus do in fact sleep well and see sleep as a refreshing escape from tinnitus. However, the prevalence of sleep disorders in chronic tinnitus patients is reported from 66% to 76%. Sleep complaints in tinnitus patients include difficulty in falling asleep, difficulty in maintaining sleep, early morning wakefulness, and non-restorative sleep with daytime sleepiness, and chronic fatigue. This can lead to mental distress, worsened anxiety and depression symptoms, and disability. Tinnitus is reported to get worse at bedtime: At bedtime, the world goes silent and that lack of noise creates confusion in the brain. The brain only knows one thing to do when that happens—create noise. It seems most likely that tinnitus does not actually wake people, but of course, it can be the first thing you notice when a natural awakening occurs.

While many hearing aids can supply a treatment for tinnitus, the relief afforded by such treatment does not necessarily continue after the hearing aid is removed. Because of the need to allow both the ear and the hearing aid to dry out (moisture in the ear canal builds up in the ear as well as in the hearing aid ear piece), and the discomfort that results from trying to sleep while wearing a such a device, it is recommended that the hearing aid be removed at night. Because of this need to remove a hearing aid at bedtime, there is a need for a bedtime device for treating HI and tinnitus and concurrently a need for a device to supply the therapeutic audio to the user that is comfortable and will not fall out because of movement during sleep.

Sleep apnea, hypopnea and snoring are also conditions that have an adverse impact on the ability of the user to both fall and stay asleep. While various devices for treating these conditions are known, there is a need for a bedtime device for treating these conditions that is comfortable and can be tolerated by a wide variety of users. There is also a need for a device that can treat a user with both (a) at least one of HI or tinnitus; and (b) at least one of sleep apnea, hypopnea and/or snoring.

### SUMMARY

The bedtime treatment of HI and tinnitus includes providing audio to a user via one or more acoustic couplers. While the following acoustic couplers are used to provide audio to a user in some embodiments, the following issues have been identified with these acoustic couplers. It has been found that earbuds (aka in-ear speakers) are prone to displacement, discomfort (lying on an earbud can be hurtful), and do not allow for the drying out of the ear canal during sleep. Headphones (aka over- or on-ear speakers) can be bulky, prone to displacement, and also uncomfortable to lay upon. Stand-alone speakers distribute (broadcast) audio widely. The potential exists that the audio from a stand-alone speaker will be overheard by the bed partner and disturb the bed partner's sleep.

A preferred solution is to supply highly directional audio targeted towards the users' head via a steerable parametric array of ultrasonic audio transducers.

In the field of acoustics, a parametric array is a nonlinear transduction mechanism that generates narrow, nearly side lobe-free beams of low frequency sound, through the mixing and interaction of high frequency sound waves, effectively overcoming the diffraction limit (a kind of spatial ‘uncertainty principle’) associated with linear acoustics. The main side lobe-free beam of low frequency sound is created as a result of nonlinear mixing of two high frequency sound beams at their difference frequency. The directivity of the radiating audible sounds is much higher than those from conventional loudspeakers.

In some embodiments, the contact-less, focused acoustic beam from a parametric array loudspeaker for the bed time delivery of audio content can be used to mitigate the loss of ambient sound due to hearing impairment (HI).

In other embodiments, the contact-less, focused acoustic beam from a parametric array loudspeaker for the bed time delivery of audio content can be used to supply therapeutic relief for those with tinnitus.

In yet another embodiment, the contact-less, focused acoustic beam from a parametric array loudspeaker for the bed time delivery of audio content can be used to supply to a user aural entertainment of the user's selection to them.

In other embodiments, the contact-less, focused acoustic beam from a parametric array loudspeaker for the bed time delivery of audio content can be used to supply a variety of masking noises to block unwelcome external sounds.

In some embodiments, the parametric array loudspeaker is manually aimed at the user's head or the pillow on which the user's head will rest during sleep, and the cross-sectional area of the focused acoustic beam output by the parametric array loudspeaker is large enough where it intersects a user's bed to accommodate a distance by which a typical user's head is expected to move while sleeping. Alternatively, since the audio beam from the parametric array loudspeaker is electronically steerable, some embodiments include one or more sensors that can be used to detect and track movement of the users' head during sleep, and the audio beam is electronically steered to compensate for such movements so that the focus of the beam is always optimized for the patient. The width of the audio beam can be more tightly focused in such embodiments.

The bedtime treatment of apnea, hypopnea and snoring includes detecting or predicting an instance of an apnea, hypopnea or snoring and providing an audio stimulus to a user, preferably via one or more contact-less, directional acoustic couplers in devices of the type discussed above for providing treatment for HI and tinnitus in order to treat the apnea, hypopnea or snoring. The use of a directional acoustic coupler to provide an acoustic stimulus upon detection or prediction of apnea, hypopnea, or snoring by the user allows for a system that can treat these breathing disorders without requiring a user to wear headphones, ear-buds or other similar devices, thereby improving user comfort and increasing the likelihood that the system will be tolerated, and thus used, by the user.

In some embodiments, a single device is configured to provide both (a) treatments for HI and/or tinnitus; and (b) treatment for apnea, hypopnea and/or snoring. This allows a user who suffers from one or more of the afflictions in group (a) and one or more of the afflictions in group (b) to be treated using a single device rather than separate devices. The single device preferably delivers contact-less acoustic neuro-modulation as described further herein.

### BRIEF DESCRIPTION OF THE DRAWINGS

This application/patent contains at least one drawing executed in color. Once this application/patent is published, copies of this patent application with color drawings will be provided by the US PTO upon request and payment of the necessary fee.

FIG. 1 is a block diagram of a controller according to a first embodiment.

FIGS. 2A and 2B illustrates the system according to an embodiment that is spoken to in the detailed description.

FIG. 3. provides an illustration as to how the system is used.

FIG. 4. provides an illustration of the difference between the spread of audio energy from a normal speaker and that of the systems array.

FIGS. 5A and 5B illustrate the directionality and the sound energy produced by an exemplary conventional speaker and an exemplary parametric loudspeaker, respectively.

### DETAILED DESCRIPTION

A method and system for the targeted, contactless application of the acoustic bedtime treatment of hearing impairment and/or tinnitus as well as user selected audio files inputted into the device from external sources (e.g., music, audio books, and other such audio files for entertainment purposes) are disclosed herein.

The system may allow the user to select one or more of a plurality of treatment functions. The user may select a hearing impairment treatment function. The hearing impairment treatment function operates in a manner similar to conventional hearing aids. Such hearing aids typically process ambient sounds received via a microphone, selectively amplify those frequencies that are attenuated due to the hearing impairment, and output to the ear of a user via a transducer such as a wired or wireless headphone, earbud, or standalone speaker as well as a contact-less, automatically directed, focused acoustic beam from a parametric array loudspeaker, which will be described in further detail below. The frequencies corresponding to the user's hearing impairment may be identified in advance in a manner known in the art. An example of this type of processing is disclosed in U.S. Pat. No. 4,508,940 and U.S. Pat. Publ. No. 20020037088A1, the entire contents of both are incorporated by reference herein. Note that the filtering and other processing described in U.S. Pat. No. 4,508,940 may be implemented using analog circuits as described therein, or may be implemented digitally by using analog-to-digital converters to sample the input sound signal from the microphone, one or more processors to perform digital filtering and other processing, and digital-to-analog converters for producing an analog output signal of the filtered ambient sounds to the user via the aforementioned transducer. Those of skill in the art will recognize that various other hearing aid processing techniques may be utilized as is known in the art. In some embodiments, the user may be able, via the device settings, to make parametric adjustments to the audio signals associated with the hearing aid function, such as amplitude and frequency emphasis/de-emphasis.

The user may also select a tinnitus treatment function. The tinnitus treatment function removes a user-defined frequency “notch” from a user selected sound files (e.g., music, audio books) or any other audio that is desired by the user that has been previously inputted into the device. “Notching” can be performed on noise (e.g., white, brown, pink or black noise) generated as a function that is resident within the device, or ambient sound inputted to the device via a microphone (which may be the same microphone discussed above in connection with the hearing impairment treatment). The user defines the frequency “notch” using controls in the device by identifying a sound frequency (which may be a simple sinusoidal frequency or a complex sound comprising two or more frequencies) that matches or at least approximates the frequency of the user's tinnitus. An exemplary process in the form of an app for identifying tinnitus frequencies is described at https://www.audionotch.com/app/tune/, the entire contents of which are incorporated by reference. U.S. Pat. No. 6,210,321 describes a similar process in which the user selectively adjusts the frequencies of two audio-frequency oscillators, one with an upper limit of 400 Hz and the other with an upper limit of 1000 Hz, in order to identify the frequencies of the tinnitus plaguing the user. The entire contents of this patent are also hereby incorporated by reference herein. The frequency or frequencies identified by the user are then filtered out from the pre-recorded sound file or ambient noise, and during treatment the resulting filtered sound file is output to the user via a transducer, which may be the same transducer discussed above in connection with the hearing impairment treatment. In some embodiments, the user may be able to make additional parametric adjustments to the audio signals associated with the tinnitus function, such as amplitude and frequency (i.e., tone) control.

The user may select a masking function wherein the user selected audio content of the device is played to assist in the auditory masking of unwelcome ambient sound. Auditory masking occurs when the perception of one sound is affected by the presence of another sound. A large-amplitude stimulus (masking sound) often makes us less sensitive to smaller stimuli of a similar nature. This is called a masking effect. The amount of masking will vary depending on the characteristics of both the target signal and the masking signal, and will also be specific to an individual listener.

The user may select an entertainment function wherein the user selected audio content of the device is played for the enjoyment of the user. Audio content can include any audio content for entertainment, such as music.

In some embodiments, the device has a controller which is a computing device that may be custom-built, using a processor (e.g., a microprocessor, microcontroller, digital signal processor, ASIC (application-specific integrated circuit), FPGA (field programmable gate array), or CPLD (complex programmable logic device), and/or combinations of the foregoing. The computing device may execute software stored internally or externally to the device. The computing device and the software act as the controller for the system. The computing device may provide for communication, which may be wired or wireless, with another device such as a PC to allow transfer of data collected by the computing device during operation so that the collected data may be displayed.

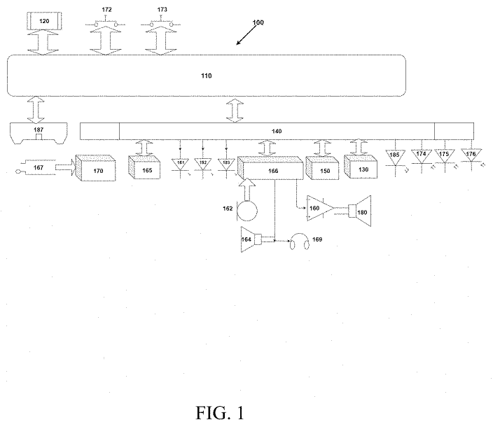

A system for implementing the functionality and treatment described above may include a computing device as described above, hereinafter referred to as a controller, and an acoustic coupler. An exemplary controller 100 is illustrated in FIG. 1. The controller 100 includes a microcontroller 110 that includes an onboard memory that may be used for, e.g., storing program instructions, and is also connected to a system memory 120 via a serial peripheral interface (SPI) or I2C bus 140, which may be used for storing collected sensor data and sound files, among other things, as discussed further below. The controller 100 may also include a digital signal processor (DSP) 130 connected to the microcontroller 110 via an I2C bus 140 in some embodiments. DSP 130 may be used for processing of therapeutic, entertainment (e.g., music, audio books) and ambient audio signals to create “notched” audio prior to being sent to the codec 166. In other embodiments, no separate DSP 130 is included and the microcontroller 110 performs this processing and other processing as discussed herein. The microcontroller 110 is connected to a wireless communication interface 150 via the I2C bus 140. The wireless communication interface 150 may be, for example, a BLUETOOTH™ interface for communication with a wireless external device (e.g., wireless earbud, wireless speaker(s), wireless headphones) that provides the audio for the therapeutic bed time treatment for the conditions described above.

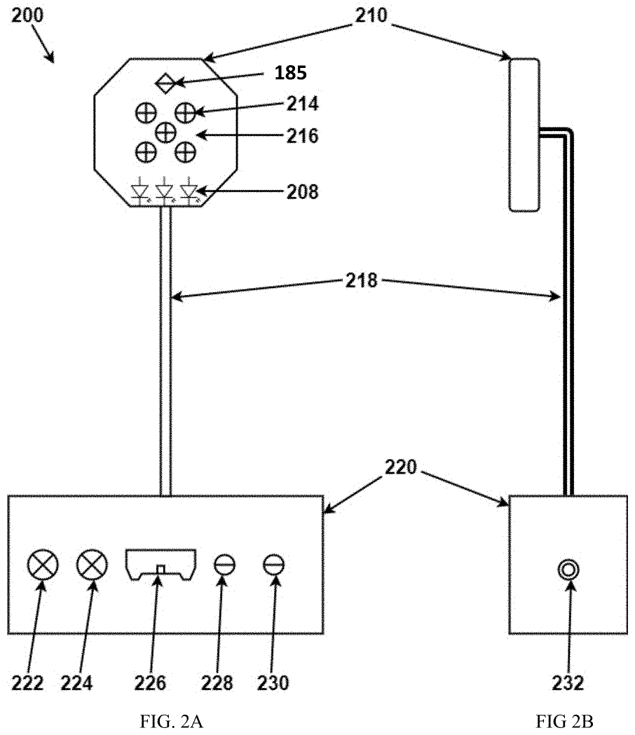

The microcontroller 110 may be connected via the I2C bus 140 to a codec 166. The codec 166 in turn may be configured to drive an amplifier 160. In turn the amplifier 160 drives an array made up of ultrasonic transducers (reference numeral 216 in FIG. 2A) that form a parametric array speaker 180.

In other embodiments the codec 166 can be configured to directly drive (power) other acoustic couplers e.g., wired earbuds (not shown), headphones 169, standalone speaker 164, etc.

Codec 166 receives ambient audio via microphone 162 in some embodiments. To provide therapy for those with HI the ambient audio collected via microphone 162 can be sent to DSP 130 for processing in order to create “notched” audio (as discussed above) prior to being sent back to the codec 166 in some embodiments. In turn codec 166 will send the audio to the acoustic coupler 164, 169 chosen by the user after the user-selected amplification factor is applied. Alternatively, if the user does not to want the ambient audio to be “notched,” then the ambient audio is processed within codec 166, and after the application of the user selected amplification factor by the amplifier 160, the audio is sent to the acoustic coupler 164, 169 chosen by the user.

A cable supplies power from an external power supply (plugged into or otherwise connected to an AC mains). The cable plugs into connector 167. In turn the voltage that enters via connector 167 goes to power supply 170. Power supply 170 converts the externally supplied voltage into different voltages required by the various electronic components in the controller 100.

The USB connector 187 is a mechanical path to establish bidirectional communication to the controller 100. This method allows a signal transmission cable to be mechanically attached to the controller 100.

Power cycling of the controller 100 is accomplished via switch 172. Power cycling of a laser diode 185 is accomplished via switch 173. Laser diode 185 is used to assist the user in the aiming of the array of ultrasonic transducers that form the parametric array speaker 180. The laser diode 185 projects a small dot of light. The dot of light from the laser diode 185 illustrates where the therapeutic audio is targeted. Ideally the target area for the therapeutic audio is the area where the head of the user will be during their sleep period, e.g., on a pillow. In other embodiments there may be more than one laser diode.

Some embodiments include sensors, such as sensors in the form of infrared optical diodes 174, 175, and 176, that are used to detect and track changes in the position of a user's head in order to steer an acoustic beam as discussed further below. The infrared optical diodes 174, 175, and 176 are spaced equally along the length of a dish (discussed further below in connection with FIG. 2A). Infrared optical diodes 174, 175, and 176 detect the heat signature (seen in the infrared portion of the light spectrum) of the users' head. The infrared optical diodes 174, 175, and 176 have a voltage output proportional to the amount of IR that they are receiving; the greater the amount of IR received, the greater the output voltage. Relative proximity of the head in relation to focal points of the infrared optical diodes 174, 175, and 176 affects the amount of received IR by the individual Infrared optical diodes. A head positioned directly under the center infrared optical diode is in the closest proximity to the center Infrared optical diode and therefore receives the most IR from the head. Those Infrared optical diodes that flank the center Infrared optical diode receive less IR; they are further away from the head. During sleep, the head repositions itself. This repositioning changes the amount of IR that is received by the Infrared optical diodes 174, 175, and 176. This in turn changes the voltages output from the infrared optical diodes 174, 175, 176. The voltages from the Infrared optical diodes can thus be used by the microcontroller 110 in some embodiments to compute the placement of the head on the pillow and track movements of the head as the head repositions while the user sleeps. In alternative embodiments, other sensors may be used. For example, thermopile sensors sold under the CoolEYE™ mark by EXCELITAS TECHNOLOGIES®, which are available in linear or two-dimensional arrays, may be used in some alternative embodiments. Other sensors and algorithms for determining and tracking a location of a user's head using the output of such sensors will be readily apparent to those of skill in the art.

The microcontroller 110 causes an ultrasonic acoustic beam output by a parametric acoustic array loudspeaker 180 comprising multiple acoustic transducers 214 (discussed further below in connection with FIGS. 2A and 2B) to remain centered on the users' head as it moves during sleep. Methods for accomplishing such movement of the beam will be discussed in further detail below. Parametric acoustic array speakers create highly directional beams of audible sound by simultaneously transmitting two ultra-sound frequencies with the audio modulating the ultrasonic frequencies. The nonlinearity of air creates both a sum and a difference frequency as the overlapping ultrasound beams propagate. Since attenuation is proportional to frequency squared, the yet higher sum frequency and original ultrasound frequencies attenuate very quickly while the low difference frequency continues to propagate through the air with similar directionality to the original ultrasound frequencies.

Steering of acoustic beams is accomplished by using phased array techniques wherein the position of the user's head as determined by the voltages from the infrared optical diodes 174-176 is used to compute the time delays for the broadcast of the individual modulated ultrasonic beams from the array 216 of ultrasonic transducers 214. By selectively controlling the time delays for each of the different beams output from transducers (i.e., based on the position of the user's head as indicated by the array of infrared optical diodes 174-176, it is possible to steer the beam formed by the combined output of the array 216 to keep the center of the beam aimed at the user's head.

In other embodiments, there may be more or fewer infrared optical diodes. In other embodiments, the position of the users' head could be determined by using ultrasonic position sensor(s), i.e., echolocation. In other embodiments, the position of the users' head could be determined by using different types of optical position sensor(s).

In still other embodiments, the shape of the beam is wide enough so that a typical user's head will remain in the acoustic beam even as the user's head shifts during sleep. In such embodiments, the laser diode 185 is used to manually aim the acoustic beam from the array 216 toward the center of the pillow on which the user's head will rest and no further adjustment to the position of the acoustic beam output by the array will be made. In such embodiments, the directionality of the acoustic beam is such that another person sleeping alongside the user in the bed will not be disturbed.

The width of the focused audio cone and the amplitude of the audio presented to the user are parameters that are adjustable by the user. FIGS. 5A and 5B illustrate the directionality and the sound energy produced by an exemplary conventional speaker and an exemplary parametric loudspeaker, respectively. Both sources in FIGS. 5A and 5B have the same apertures of 10 cm in radius, radiating sounds at a frequency of 2 kHz. The primary frequencies are 40 and 42 kHz.

In practice, a user would typically set the sound energy directly focused at the user's head to be in the range of 30 dB and 60 dB, inclusive. This would result in a sound of 20 dB being received at another sleeper's head approximately 0.5 meters from the head of the user being treated.

LED 181 indicates when lit that the device 100 is active (on). LED 182 indicates when lit that the laser diode 183 is active (on).

An embodiment of a system 200 is illustrated in FIGS. 2A and 2B. FIG. 2A provides a front view of the system 200, and FIG. 2B provides a side view of the system 200. A dish 210 holds an array 216 made up of ultrasonic transducers 214. In FIG. 2A, the array 216 is made up of five ultrasonic transducers 214. In other embodiments, the array 216 may have more, or less, ultrasonic transducers 214 and in different configurations (positions).

The laser diode 185 is attached to the dish 210. The dish 210 holds an array 208 of infrared optical diodes, which includes the optical diodes 174-176 discussed above. In FIG. 2A there are three infrared optical diodes in the array 208 (i.e., diodes 174, 175 and 176). In other embodiments there may be more or fewer Infrared optical diodes in the array 208.

A gooseneck (flexible tubing) 218 supplies the support for dish 210 and allows the user to initially position (aim) the dish 210 so as to optimize the targeted area for the audio. In other embodiments, the dish 210 is supported by an articulated arm. Gooseneck 218 attaches to the dish 210 and an enclosure 220 that houses the controller 100. Wiring from the controller 100, as discussed above, travels through the hollow core of the gooseneck 218 to the dish 210 and from there to the array 216 of the five ultrasonic transducers 214 and the laser diode 212.

As discussed above, some embodiments may determine the position of a user's head and adjust the position of the acoustic beam output by the ultrasonic transducers 214 so that the beam remains centered on the user's head. The user's head rests on a surface of a pillow. The surface of a pillow can be considered to be a two-dimensional object; with vertical (Y-axis) and horizontal (X-axis) dimensions (positions) on a plane. In some embodiments, IR diodes 174-176 perceive the heat signature from the human head. Because the IR 174-176 diodes are spaced apart along the x-axis, when the user's head moves on the x-axis of the pillow, the IR diode that is closest to that IR signature will output the greatest voltage. The relative strength of the outputted voltages from the IR diodes 174-176 is used to steer the beam, e.g., by computing the position of the head on the pillow. This process can be repeated at a periodic rate, e.g., once per second. In some embodiments, each newly calculated position is used to steer the beam. In other embodiments, each newly calculated position is used to update a filtered position value.

When the position of the user's head is known, some embodiments adjust the position of the beam (i.e., steer the beam) to keep the beam centered on the user's head as it moves during sleep. In some embodiments, the steering of the acoustic beam is done purely electronically. In some embodiments, the position information is used to compute the order in which the discreet ultrasonic transducers produce ultrasonic beams as well as their relative loudness (amplitude). In this way the focus of the combined beams is steered to the current location of the user's head.

In other embodiments, adjusting the positioning of the focused acoustic beam is performed via electro-mechanical means. Servo-mechanisms similar to those used in articulated robots, SCARA (selective compliance assembly robot arm) robots, delta robots, or a cartesian robots may physically re-position the dish 210 so that the focus and the tracking of the acoustic beam remains on the users' head. In this and other embodiments, the dish 210 may be separate from the controller 100. Power, audio, and control signals may be supplied to dish 210 by wires from the enclosure 220. The dish 210 may be mechanically attached to other surfaces, such as a wall or headboard, for support and positioning.

The enclosure 220 contains the controller 100 as discussed above. In this exemplary embodiment, the enclosure 220 has a switch 222. Switch 222 controls the voltage supplied to the entire system 200. The enclosure 220 may have a second switch 224, which is used to control the voltage supplied to the laser diode 185. The enclosure 220 may include a USB connector 226. USB connector 226 is discussed above in conjunction with controller 100. The enclosure 220 may have an LED 228 that, when lit, indicates that the system 200 has power. The enclosure 220 may include another LED 230 that, when lit, indicates that the laser diode 185 has power.

In other embodiments, system status information, user-controlled configuration options (e.g., among others, “notching” process of selected audio files, enabling of HI and/or tinnitus treatment, choice of audio files and/or noise generation, sound file amplitude, timer functions, etc.) and data, such as that supplied by LED 228 and LED 230, can be implemented by other means, such as a display (e.g., Liquid Crystal (LCD), ELED, QLED, OLED, AMOLED, LED) or any other display technology that is current or may be developed in the future.

In other embodiments, system status information, user-controlled configuration options (e.g., among others, “notching” process of selected audio files, enabling of HI and/or tinnitus treatment, choice of audio files and/or noise generation, sound file amplitude, timer functions, etc.) and data, such as that supplied by LED 228 and LED 230, can be implemented by other means, such as an application running on a smartphone (not shown) or via a remote control (not shown).

In other embodiments system controls, such as switch 222, could be implemented by other methods (e.g., touch display, mechanical and electronic touch buttons, sliders).

Those persons who are skilled in the art will understand that the embodiments of system controls and displays are the means by which the user can select functions such as the creation of “notched” audio files, recordings from external sources of audio files, selection of the order in which audio files are played, the amplitude that “notched” or unprocessed audio files are played at, the amplitude of the ambient noise that is supplied via the device, establishing timers that will halt the playing of audio, the selection of the type of noise to be played. This paragraph is not the inclusive list of user setting that may be included.

The enclosure 220 may have connector 232. A cable (not shown) supplies power from an external power supply such as an AC mains. The cable plugs into connector 232, which is connected to internal connector 167 which in turn is connected to the power supply 170 of controller 100, as discussed above.

The enclosure 220 in the exemplary illustration has a flat bottom. In another embodiment, a table-style clamp may be added to the bottom of the enclosure 220 in order to allow the system to be attached to a bed headboard. In another embodiment, flanges may be added to the bottom of the enclosure 220 in order to allow the system to be attached to a wall above the bed. In other embodiments, attachment of enclosure 220 to a surface can be accomplished via a number of means known in the art.

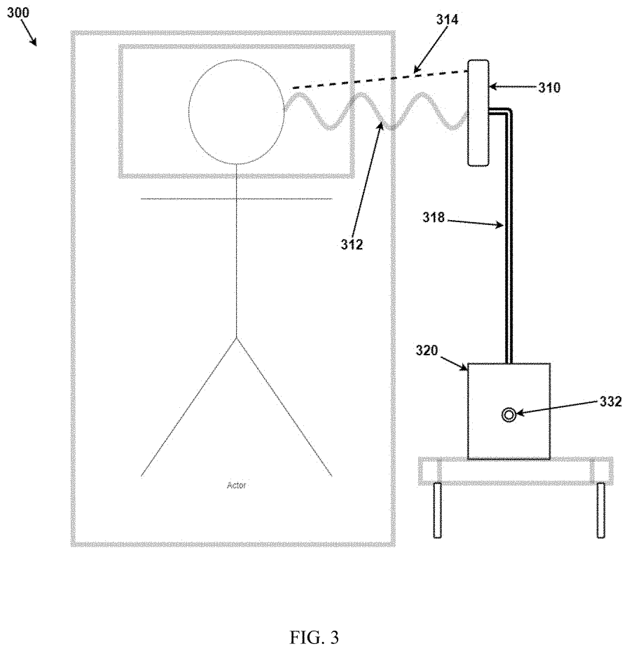

FIG. 3 provides an illustration as to how the system may be used. In a preferred embodiment, the system is mounted on a bed side table with the enclosure 320 resting on its surface. The user manipulates the gooseneck 318 and dish 310 so that the beam of light 314 from the laser diode falls upon the area where the users head and ears will be positioned during sleep. In this way, the audio 312 supplied by the ultrasonic transducers forming the array (as shown in FIG. 2A as array 216 made up of ultrasonic transducers 214), is also directed towards the user's head and ears.

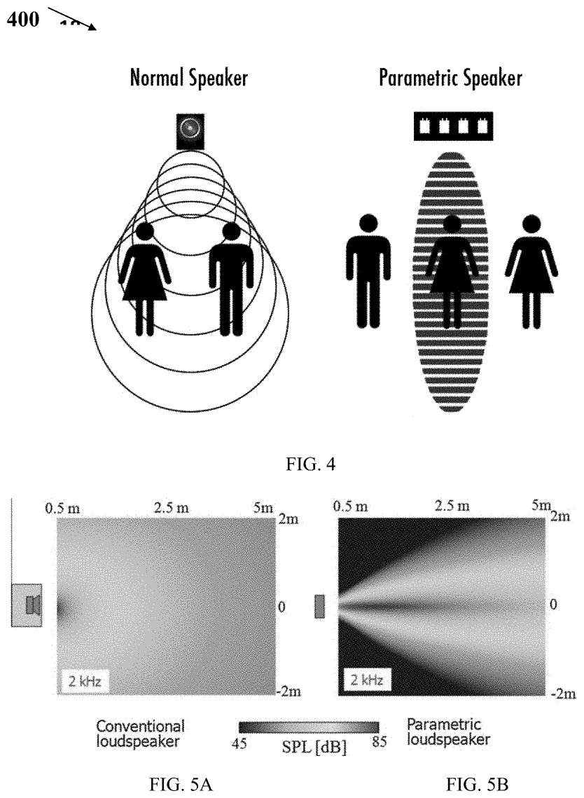

FIG. 4 illustrates the difference between the spread of audio energy from a normal speaker and that of the array used in some embodiments (in FIG. 4, the array is referred to as a “Parametric Speaker”). As shown, the audio energy from a normal speaker is wide spread an opposed to that of the parametric speaker.

As discussed above, a parametric array loudspeaker is preferably used as the acoustic coupler. It's important to note that different names for this technology is used in literature describing other projects (commercial, scientific or of other nature), uses, products and implementations Some of this other terminology includes “parametric loudspeakers”, “parametric speakers”, “parametric acoustic array”, “parametric array”, “parametric audio system”, “hypersonic sound”, “beam of sound”, “audible sound beams”, “super directional sound beams”, “super directional loudspeaker”, “focused audio”, “audio spotlight”, “phased array sound system”, “digital array speaker”, “Sigma-Delta loudspeaker array, “digital loudspeaker array”, “Digital Transducer Array loudspeaker”, and “Parametric Digital Transducer Array Loudspeaker”, among others.

In other embodiments, the loudspeaker array may consist of a number of acoustic transducers (e.g., electro-mechanical speakers, MEMs speakers, ultrasonic transducers (e.g., piezo-electric transducers)) placed into a pattern.

In another embodiment, the system of FIGS. 1 and 2A-2B used for treating HI and/or tinnitus is configured for the treatment of apnea, hypopnea and/or snoring. Detailed descriptions of techniques for treating sleep apnea, hypopnea and/or snoring is found in U.S. Pat. No. 11,089,994, U.S. Pat. Pub. Nos. 2015/0173672, 2014/0051938, 2005/0197588, 2016/0045154, and 2009/0076405, and WIPO Pub. No. WO 96/28093. The entire contents of all of these documents are hereby incorporated herein by reference. In some preferred embodiments, the disclosures of those documents are modified by using the parametric array of ultrasonic audio transducers to produce a focused acoustic beam directed at the user's head as discussed above for providing the acoustic neuromodulation stimulus to the user in place of a stimulus delivered via headphone, earbuds, or other devices worn on the user's head or placed into or over the user's ears. This change increases user comfort as discussed above. The microphone used in such embodiments may be the same microphone 162 discussed above for detecting ambient sounds or may be an additional microphone. Such embodiments may also utilize a plethysmograph for the detection or prediction of apneas as discussed in the aforementioned U.S. Pat. No. 11,089,994. Using the same microphone 162 both to detect ambient sounds and to monitor the user for apneas, hypopneas and/or snoring reduces cost, but in some embodiments a separate microphone is preferable as the use of a separate microphone to detect or predict apneas, hypopneas and/or snoring is preferable as such a separate microphone may be more appropriately positioned for detecting or predicting these afflictions in the user rather than the user's bed partner.

In those embodiments that are configured only for the treatment of apnea or hypopnea (and not HI or tinnitus), the controller 110 may be configured to detect or predict the occurrence of an instance of apnea, hypopnea or snoring using just the signal detected from the microphone and, upon such direction, cause the directional array 216 of transducers to output a stimulus that terminates the apnea, hypopnea or snoring. In other embodiments, sensors in addition to the microphone, such as the plethysmograph disclosed in U.S. Pat. No. 11,089,994 may also be used to determine when to apply an acoustic stimulus as described therein.

In multi-treatment embodiments configured to treat HI and/or tinnitus in addition to hypopnea, apnea and/or snoring, the controller 110 is configured to apply the HI and/or tinnitus treatments discussed above until the user falls asleep. Once the user has fallen asleep, the provision of the tinnitus treatment (if the user requires such treatment) ceases, while treatment (such as, e.g., amplification of ambient sounds as described above) for HI continues (if needed). At the same time, the controller 110 monitors the output of the microphone to detect or predict instances of apnea, hypopnea and/or snoring, and, in response, to cause an audio stimulation to be applied via the acoustic coupler to prevent or terminate the apnea, hypopnea and/or snoring using the techniques discussed above.

Although the invention has been described in connection with certain preferred embodiments, it should be understood that various modifications, additions and alterations may be made to the invention by one skilled in the art without departing from the spirit and scope of the invention as defined in the appended claims.

What is claimed is:

1. A method for treating hearing impairment and/or tinnitus of a user in a bed, the method comprising:
aiming a parametric array speaker in a direction of a head of a user in a bed; and
delivering a focused acoustic beam from the parametric array speaker to the user's head.

2. The method of claim 1, further comprising:
tracking a movement of the user's head; and
electronically steering the focused acoustic beam to compensate for the movement of the user's head.

3. The method of claim 1, further comprising manually aiming the parametric array speaker.

4. The method of claim 3, wherein the parametric array is manually aimed using a visual indicator output by a laser diode mounted to the parametric array loudspeaker.

5. The method of claim 1, further comprising the steps of:
detecting ambient sound using a microphone, and
driving the parametric array speaker using the ambient sound detected by the microphone.

6. The method of claim 5, further comprising the step of filtering the ambient sound detected by the microphone to remove a frequency notch corresponding to the user's tinnitus frequencies, wherein the filtered ambient sound is used to drive the parametric speaker array.

7. The method of claim 6, further comprising the step of inputting an indication of the user's tinnitus frequencies from the user, wherein the frequency notch corresponds to the indication of the user's tinnitus frequencies from the user.

8. The method of claim 1, further comprising the step of:
driving the parametric array speaker using a pre-recorded sound file.

9. The method of claim 8, further comprising the stop of accepting a selection of a pre-recorded sound file from a user, wherein the pre-recorded sound file selected by the user is used to drive the parametric array speaker.

10. The method of claim 1, further comprising the step of driving the parametric array speaker with a noise signal.

11. The method of claim 10, wherein the noise signal does not include noise in a frequency notch corresponding to the user's tinnitus frequencies.

12. A device comprising:
a parametric array speaker; and
a controller connected to drive the parametric array speaker with a notched audio signal, wherein the notched audio signal does not include frequencies in a frequency notch corresponding to a user's tinnitus frequencies.

13. The device of claim 12, further comprising a plurality of sensors configured to detect radiation from the user's head;
wherein the controller is configured to determine a position of the user's head using information from the sensors and steer an output of the parametric array speaker toward the user's head.

14. The device of claim 12, further comprising a microphone configured to detect ambient sound, wherein the controller is configured to filter the ambient sound detected by microphone to remove a frequency notch corresponding to the user's tinnitus frequencies to create the notched audio signal used to drive the parametric array speaker.

15. A method for treating apnea, hypopnea and/or snoring, the method comprising:
monitoring an output of a microphone to detect an instance of an apnea, hypopnea and/or snoring by the user; and
in response to detecting or predicting an instance of an apnea, hypopnea and/or snoring by the user, delivering an audio stimulus via a focused acoustic beam from a parametric array speaker aimed in a direction of the head of the user.
